(19) United States
(12) Patent Application Publication
(10) Pub. No.: US 2017/0029505 A1
(43) Pub. Date: Feb. 2, 2017

(54) MULTIMERIC FC PROTEINS

(71) Applicant: UCB BIOPHARMA SPRL, Brussels (BE)

(72) Inventors: Robert Anthony GRIFFIN, Slough (GB); David Paul HUMPHREYS, Slough (GB); Shirley Jane PETERS, Slough (GB)

(73) Assignee: UCB BIOPHARMA SPRL, Brussels (BE)

(21) Appl. No.: 15/303,625

(22) PCT Filed: Apr. 16, 2015
(86) PCT No.: PCT/EP2015/058338

§ 371 Date: Oct. 12, 2016

(30) Foreign Application Priority Data
Apr. 16, 2014 (GB) 1406894.4
Jul. 16, 2014 (GB) 1412649.4

(51) Int. Cl. C07K 16/28; C07K 16/30

(52) U.S. Cl. CPC C07K 16/283; C07K 16/30; C07K 16/3015; C07K 16/3023; C07K 16/303; C07K 16/3038; C07K 16/3053; C07K 16/3061; C07K 16/3069; C07K 2317/53; C07K 2317/72; C07K 2317/622; C07K 2317/569; C07K 2319/30; C07K 2317/22

(57) ABSTRACT

The invention relates to multimeric proteins which bind to human Fc receptors. The invention also relates to therapeutic compositions comprising the proteins, and their use in the treatment of immune and other disorders.

The invention relates to multimeric proteins which bind to human Fc-receptors. The invention also relates to therapeutic compositions comprising the multimeric proteins, and their use in the treatment of immune and other disorders.

### BACKGROUND

Immune disorders encompass a wide variety of diseases with different signs, symptoms, etiologies and pathogenic mechanisms. Many of these diseases are characterised by the active involvement of pathogenic antibodies and/or pathogenic immune complexes. In some diseases such as ITP (variably called immune thrombocytopenia, immune thrombocytic purpura, idiopathic thrombocytopenic purpura) the target antigens for the pathogenic antibodies (Hoemberg, Scand H J Immunol, Vol 74(5), p489-495, 2011) and disease process are reasonably well understood. Such immune disorders are often treated with a variety of conventional agents, either as monotherapy or in combination. Examples of such agents are corticosteroids, which are associated with numerous side effects, intravenous immunoglobulin (IVIG) and anti-D.

Antibodies, often referred to as immunoglobulins, are Y-shaped molecules comprising two identical heavy (H) chains and two identical light (L) chains, held together by interchain disulphide bonds. Each chain consists of one variable domain (V) that varies in sequence and is responsible for antigen binding. Each chain also consists of at least one constant domain (C). In the light chain there is a single constant domain (CL). In the heavy chain there are at least three, sometimes four constant domains, depending on the isotype (CH1, CH2, CH3, CH4). IgG, IgA and IgD have three heavy chain constant domains; IgM and IgE have four.

In humans there are five different classes or isotypes of immunoglobulins termed IgA, IgD, IgE, IgG and IgM. All these classes have the basic four-chain Y-shaped structure, but they differ in their heavy chains, termed α, δ, ε, γ and μ respectively. IgA can be further subdivided into two subclasses, termed IgA1 and IgA2. There are four sub-classes of IgG, termed IgG1, IgG2, IgG3 and IgG4.

The Fc-domain of an antibody typically comprises at least the last two constant domains of each heavy chain which dimerise to form the Fc domain. The Fc domain is responsible for providing antibody effector functions, including determining antibody half-life, principally through binding to FcRn, distribution throughout the body, ability to fix complement, and binding to cell surface Fc receptors.

The differences between antibody isotypes are most pronounced in the Fc-domains, and this leads to the triggering of different effector functions on binding to antigen. Structural differences also lead to differences in the polymerisation state of the antibodies. Thus IgG, IgE and IgD are generally monomeric whereas IgM occurs as both a pentamer and a hexamer, IgA occurs predominantly as a monomer in serum and as a dimer in sero-mucous secretions.

Intravenous immunoglobulin (IVIG) is the pooled immunoglobulin from thousands of healthy blood donors. IVIG was initially used as an IgG replacement therapy to prevent opportunistic infections in patients with low IgG levels (reviewed in Baerenwaldt , Expert Rev Olin lmmunol, Vol 6(3), p425-434, 2010). After discovery of the anti-inflammatory properties of IVIG in children with ITP (Imbach, Helv Paediatri Acta, Vol 36(1), p81-86, 1981), IVIG is now licensed for the treatment of ITP, Guillain-Barré syndrome, Kawasaki disease, and chronic inflammatory demyelinating polyneuropathy (Nimmerjahn, Annu Rev Immunol, Vol 26, p513-533, 2008).

In diseases involving pathogenic immune complexes it has been proposed that a minority fraction of the component immunoglobulin fraction is disproportionately effective. It is observed that traces (typically 1-5%) of IgG are present in multimeric forms within IVIG. The majority of this multimeric fraction is thought to be dimer with smaller amounts of trimer and higher forms. It has also been proposed that additional dimers may form after infusion by binding of recipient anti-idiotype antibodies. One theory is that these multimeric forms compete against immune complexes for binding to low affinity FCγ receptors due to their enhanced avidity (Augener, Blut, Vol 50, p249-252, 1985; Teeling, Blood Vol 98(4), p1095-1099, 2001; Machino, Y., Olin Exp lmmunol, Vol 162(3), p415-424, 2010; Machino, Y. et al., BBRC, Vol 418, p748-753, 2012). Another theory is that sialic acid glycoforms of IgG within IVIG, especially the presence of higher levels of α2-6 sialic acid forms, cause an alteration in FCγ receptor activation status (Samuelsson, Science, Vol 291, p484-486, 2001; Kaneko, Science, Vol 313, p670-673, 2006; Schwab, European J Immunol Vol 42, p826-830, 2012; Sondermann, PNAS, Vol 110(24), p9868-9872, 2013).

In diseases involving pathogenic antibodies it has been proposed that the very large dose of IVIG administered to humans (1-2 g/kg) effectively overrides the normal IgG homeostasis mechanism performed by FcRn. Effectively a large dilution of recipient IgG by donor IVIG results in enhanced catabolism and a shorter serum half-life of patient pathogenic antibodies. Other proposed mechanisms for the efficacy include anti-idiotypic neutralisation of pathogenic antibodies and transient reductions in complement factors (Mollnes, Mol Immunol, Vol 34, p719-729, 1997; Crow, Transfusion Medicine Reviews, Vol 22(2), p103-116, 2008; Schwab, I. and Nimmerjahn, F. Nature Reviews Immunology, Vol 13, p176-189, 2013).

There are significant disadvantages to the clinical use of IVIG. IVIG has variable product quality between manufacturers due to inherent differences in manufacturing methods and donor pools (Siegel, Pharmacotherapy Vol 25(11) p78S-84S, 2005). IVIG is given in very large doses, typically in the order of 1-2 g/kg. This large dose necessitates a long duration of infusion, (4-8 hours, sometimes spread over multiple days), which can be unpleasant for the patient and can result in infusion related adverse events. Serious adverse events can occur, reactions in IgA deficient individuals being well understood. Cytokine release can also be observed in patients receiving IVIG but this is largely minimised by careful control of dose and infusion rate. As a consequence of the large amounts used per patient and the reliance on human donors, manufacture of IVIG is expensive and global supplies are severely limited.

Collectively the disadvantages of IVIG mean that there is need for improvement in terms of clinical supply, administration and efficacy of molecules able to interfere with the disease biology of pathogenic antibodies and pathogenic immune complexes.

Polymeric proteins have been described in the prior art in which the carboxyl-terminal tailpiece from either IgM or IgA was added to the carboxyl-termini of whole IgG3 molecule constant regions to produce recombinant IgM-like IgG3. (Sorensen V. et al, J lmmunol, Vol 156, p2858-2865, 1996). The IgG3 molecules were additionally modified by substituting the leucine residue at position 309 with a cysteine residue (L309C). In some experiments, the tailpiece was omitted and the IgG3 molecules were modified with L309C only. The IgG3 molecules studied were intact immunoglobulin molecules. In contrast, the multimeric proteins of the present invention do not comprise the first heavy chain constant domain, CH1.

In the present invention we provide improved multimeric proteins which resolve many of the disadvantages of IVIG. The proteins may be produced in large quantities, under carefully controlled conditions, eliminating the problems of limited supply and variable quality. Furthermore, the improved multimeric proteins of the present invention have therapeutic applications in other disorders as described herein.

### DESCRIPTION OF THE INVENTION

The multimeric proteins of the invention have been collectively named “Fc-multimers” and the two terms are used interchangeably herein

Unless defined otherwise, all technical and scientific terms used herein have the same meaning as commonly understood by one of skill in the art to which this invention belongs. All publications and patents referred to herein are incorporated by reference.

It will be appreciated that any of the embodiments described herein may be combined.

In the present specification the EU numbering system is used to refer to the residues in antibody domains, unless otherwise specified. This system was originally devised by Edelman et al, 1969 and is described in detail in Kabat et al, 1987.

Edelman et al., 1969; “The covalent structure of an entire γG immunoglobulin molecule,” PNAS Biochemistry Vol.63 pp78-85.

Kabat et al., 1987; in Sequences of Proteins of Immunological Interest, US Department of Health and Human Services, NIH, USA.

Where a position number and/or amino acid residue is given for a particular antibody isotype, it is intended to be applicable to the corresponding position and/or amino acid residue in any other antibody isotype, as is known by a person skilled in the art.

The present invention provides a multimeric protein comprising two or more polypeptide monomer units;

wherein each polypeptide monomer unit comprises an antibody Fc-domain comprising two heavy chain Fc-regions,

wherein each heavy chain Fc-region comprises a cysteine residue at position 309 which causes the monomer units to assemble into a multimer,

and wherein each polypeptide monomer unit does not comprise a CH1 domain or a tailpiece.

CH1 domain refers to the first antibody heavy chain constant domain.

The inventors have found that antibody Fc domains can be multimerised into multivalent forms by engineering the presence of a cysteine residue at position 309. They have observed that the higher order valency forms result in higher avidity binding for Fc-receptors (FcR) and also elevated levels of cytokine release in human whole blood. Higher valency is desirable in certain immune indications such as ITP, GBS and CIDP, to achieve effective blockade of FcγR from pathogenic antibodies and immune complexes. Elevated cytokine release may be useful or detrimental depending upon the intended clinical use and molecular target. In targeted cell killing applications such as cancer and other proliferative disorders, elevated cytokine levels may be advantageous. Fusion of an antigen targeting moiety such as scFv, single domain antibody (for example vL, vH, vHH, shark VNAR, camelid v-region), engineered SH3 domain, or DARPin, at the N- or C-terminus of the multimeric proteins of the invention can target the multimeric Fc protein to the target cell and elicit killing through well described effector functions such as CDC, ADCC and ADCP.

These functions can be enhanced by the increased avidity of FcγR binding. In addition, high local levels of cytokines such as IFNγ and TNFα which have cytotoxic effects can augment cell killing. Local cytokines may also elicit immune cell infiltration and hence augment anti-target responses. In vaccine applications, fusion of an antigen moiety such as allergen peptide, tumour antigen or similar at the N- or C-terminus of the multimeric proteins of the invention can target the multimeric Fc protein to target cells involved in antigen presentation. Dendritic cells, macrophages, monocytes and neutrophils are all capable of antigen uptake, digestion and presentation through MHC-I or MHC-II to T-cells. Hence, enhanced targeting of antigen to antigen presenting cells through increased avidity of binding to FcγR is desirable. Additionally, increased local cytokine production might further increase the immune response due to activation of or infiltration of activated immune cells. Further improvements to targeting in cell killing and vaccine approaches may be made by Fc-mutations which influence FcγR or FcRn binding.

Thus, in one example, the multimeric proteins of the present invention further comprise a fusion partner. The term ‘fusion partner’ may refer to an antigen targeting moiety selected from the group comprising scFv, single domain antibody (for example vL, vH, vHH, shark VNAR, camelid v-region), engineered SH3 domain, or DARPin. Alternatively, the fusion partner may be an antigen (for example an allergen peptide or tumour antigen), pathogen-associated molecular pattern (PAMP), drug, ligand, receptor, cytokine or chemokine.

Examples of tumour antigens include:
a Mage gene product, for example MAGE tumour antigen, for example, MAGE 1, MAGE 2, MAGE 3, MAGE 4, MAGE 5, MAGE 6, MAGE 7, MAGE 8, MAGE 9, MAGE 10, MAGE 11 or MAGE 12. The genes encoding these MAGE antigens are located on chromosome X and share with each other 64 to 85% homology in their coding sequence (De Plaen, 1994). These antigens are sometimes known as MAGE Al, MAGE A2, MAGE A3, MAGE A4, MAGE A5, MAGE A6, MAGE A7, MAGE A8, MAGE A9, MAGE A 10, MAGE Al 1 and/or MAGE A12 (The MAGE A family). In one embodiment, the antigen is MAGE and/or an antigen from one of two further MAGE families may be used: the MAGE B and MAGE C group. The MAGE B family includes MAGE B 1 (also known as MAGE Xp 1, and DAM 10), MAGE B2 (also known as MAGE Xp2 and DAM 6) MAGE B3 and MAGE B4-the Mage C family currently includes MAGE C1 and MAGE C2;

cancer testis antigens such as PRAME, LAGE 1, LAGE 2;

SSX-2, SSX-4, SSX-5, NA17, MELAN-A, Tyrosinase, LAGE-I, NY-ESO-I, PFRAME; P790, P510, P835, B305D, B854, 01491, 01584, and 01585. In one embodiment, the antigen may comprise or consist of P501S (also known as prostein), and

WT-I expressed by the Wilm's tumor gene, or its N-terminal fragment WT-IF comprising about or approximately amino acids 1-249;

the antigen expressed by the Her-2/neu gene, or a fragment thereof.

Said fusion partner, where present, is fused to the N-terminus and/or the C-terminus of the or each heavy chain Fc-region. The fusion partner may be fused directly to the N- and/or C-terminus of the heavy chain Fc-region. Alternatively it may be fused indirectly by means of an intervening amino acid sequence, which may include a hinge, where present. For example, a short linker sequence may be provided between the fusion partner and the heavy chain Fc-region.

In certain applications such as treatment of immune disorders a fusion partner may not be required. Thus, in one example, the proteins of the present invention do not comprise a fusion partner.

Each polypeptide monomer unit of the multimeric protein of the present invention comprises an antibody Fc-domain.

The antibody Fc-domain of the present invention may be derived from any suitable species. In one embodiment the antibody Fc-domain is derived from a human Fc-domain.

The antibody Fc-domain may be derived from any suitable class of antibody, including IgA (including subclasses IgA1 and IgA2), IgD, IgE, IgG (including subclasses IgG1, IgG2, IgG3 and IgG4), and IgM. In one embodiment, the antibody Fc-domain is derived from IgG1, IgG2, IgG3 or IgG4. In one embodiment the antibody Fc-domain is derived from IgG1. In one embodiment the antibody Fc-domain is derived from IgG4.

The antibody Fc-domain comprises two polypeptide chains, each referred to as a heavy chain Fc-region. The two heavy chain Fc-regions dimerise to create the antibody Fc-domain. Whilst the two heavy chain Fc-regions within the antibody Fc-domain may be different from one another it will be appreciated that these will usually be the same as one another. Hence where the term ‘the heavy chain Fc-region’ is used herein below this is used to refer to the single heavy chain Fc-region which dimerises with an identical heavy chain Fc-region to create the antibody Fc-domain.

Typically each heavy chain Fc-region comprises or consists of two or three heavy chain constant domains.

In native antibodies, the heavy chain Fc-region of IgA, IgD and IgG is composed of two heavy chain constant domains (CH2 and CH3) and that of IgE and IgM is composed of three heavy chain constant domains (CH2, CH3 and CH4). These dimerise to create an Fc-domain.

In the present invention, the heavy chain Fc-region may comprise heavy chain constant domains from one or more different classes of antibody, for example one, two or three different classes.

In one embodiment the heavy chain Fc-region comprises CH2 and CH3 domains derived from IgG1.

In one embodiment the heavy chain Fc-region comprises CH2 and CH3 domains derived from IgG2.

In one embodiment the heavy chain Fc-region comprises CH2 and CH3 domains derived from IgG3.

In one embodiment the heavy chain Fc-region comprises CH2 and CH3 domains derived from IgG4.

In one embodiment the heavy chain Fc-region comprises a CH4 domain from IgM. The IgM CH4 domain is typically located at the C-terminus of the CH3 domain.

In one embodiment the heavy chain Fc-region comprises CH2 and CH3 domains derived from IgG and a CH4 domain derived from IgM.

It will be appreciated that the heavy chain constant domains for use in producing a heavy chain Fc-region of the present invention may include variants of the naturally occurring constant domains described above. Such variants may comprise one or more amino acid variations compared to wild type constant domains. In one example the heavy chain Fc-region of the present invention comprises at least one constant domain which varies in sequence from the wild type constant domain. It will be appreciated that the variant constant domains may be longer or shorter than the wild type constant domain. Preferably the variant constant domains are at least 50% identical or similar to a wild type constant domain. The term “identity”, as used herein, indicates that at any particular position in the aligned sequences, the amino acid residue is identical between the sequences. The term “similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).

Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). In one example the variant constant domains are at least 60% identical or similar to a wild type constant domain. In another example the variant constant domains are at least 70% identical or similar. In another example the variant constant domains are at least 80% identical or similar. In another example the variant constant domains are at least 90% identical or similar. In another example the variant constant domains are at least 95% identical or similar.

IgM and IgA occur naturally in humans as covalent multimers of the common H₂L₂ antibody unit. IgM occurs as a pentamer when it has incorporated a J-chain, or as a hexamer when it lacks a J-chain. IgA occurs as monomer and dimer forms. The heavy chains of IgM and IgA possess an 18 amino acid extension to the C-terminal constant domain, known as a tailpiece. The tailpiece includes a cysteine residue that forms a disulphide bond between heavy chains in the polymer, and is believed to have an important role in polymerisation. The tailpiece also contains a glycosylation site. The multimeric proteins of the present invention do not comprise a tailpiece.

Each heavy chain Fc-region of the present invention may optionally possess a native or a modified hinge region at its N-terminus.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc-region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO9915549, WO2005003170, WO2005003169, WO2005003170, WO9825971 and WO2005003171 and these are incorporated herein by reference.

Examples of suitable hinge sequences are shown in Table 1.

In one embodiment, the heavy chain Fc-region possesses an intact hinge region at its N-terminus.

In one embodiment the heavy chain Fc-region and hinge region are derived from

IgG4 and the hinge region comprises the mutated sequence CPPC (SEQ ID NO: 9). The core hinge region of human IgG4 contains the sequence CPSC (SEQ ID NO: 10) compared to IgG1 which contains the sequence CPPC. The serine residue present in the IgG4 sequence leads to increased flexibility in this region, and therefore a proportion of molecules form disulphide bonds within the same protein chain (an intrachain disulphide) rather than bridging to the other heavy chain in the IgG molecule to form the interchain disulphide. (Angel S. et al, Mol lmmunol, Vol 30(1), p105-108, 1993). Changing the serine residue to a proline to give the same core sequence as IgG1 allows complete formation of inter-chain disulphides in the IgG4 hinge region, thus reducing heterogeneity in the purified product. This altered isotype is termed IgG4P.

TABLE 1
Hinge sequences

| Hince | Sequence |
|---|---|
| Human IgA1 | VPSTPPTPSPSTPPTPSPS SEQ ID NO: 1 |
| Human IgA2 | VPPPPP SEQ ID NO: 2 |
| Human IgD | ESPKAQASSVPTAQPQAEGSLAKATTAPATTRN |
| | TGRGGEEKKKEKEKEEQEERETKTP |
| | SEQ ID NO: 3 |
| Human IgG1 | EPKSCDKTHTCPPCP SEQ ID NO: 4 |
| Human IgG2 | ERKCCVECPPCP SEQ ID NO: 5 |
| Human IgG3 | ELKTPLGDTTHTCPRCPEPKSCDTPPPCPRCPE |
| | PKSCDTPPPCPRCPEPKSCDTPPPCPRCP |
| | SEQ ID NO: 6 |
| Human IgG4 | ESKYGPPCPSCP SEQ ID NO: 7 |
| Human IgG4(P) | ESKYGPPCPPCP SEQ ID NO: 8 |
| Recombinant v1 | CPPC SEQ ID NO: 9 |
| Recombinant v2 | CPSC SEQ ID NO: 10 |
| Recombinant v3 | CPRC SEQ ID NO: 11 |
| Recombinant v4 | SPPC SEQ ID NO: 12 |
| Recombinant v5 | CPPS SEQ ID NO: 13 |
| Recombinant v6 | SPPS SEQ ID NO: 14 |
| Recombinant v7 | DKTHTCAA SEQ ID NO: 15 |
| Recombinant v8 | DKTHTCPPCPA SEQ ID NO: 16 |
| Recombinant v9 | DKTHTCPPCPATCPPCPA SEQ ID NO: 17 |
| Recombinant v10 | DKTHTCPPCPATCPPCPATCPPCPA |
| | SEQ ID NO: 18 |
| Recombinant v11 | DKTHTCPPCPAGKPTLYNSLVMSDTAGTCY |
| | SEQ ID NO: 19 |
| Recombinant v12 | DKTHTCPPCPAGKPTHVNVSVVMAEVDGTCY |
| | SEQ ID NO: 20 |
| Recombinant v13 | DKTHTCCVECPPCPA SEQ ID NO: 21 |
| Recombinant v14 | DKTHTCPRCPEPKSCDTPPPCPRCPA |
| | SEQ ID NO: 22 |
| Recombinant v15 | DKTHTCPSCPA SEQ ID NO: 23 |

The multimeric protein of the invention may comprise two, three, four, five, six, seven, eight, nine, ten, eleven or twelve or more polypeptide monomer units. Such proteins may alternatively be referred to as a dimer, trimer, tetramer, pentamer, hexamer, heptamer, octamer, nonamer, decamer, undecamer, dodecamer, etc., respectively.

In one embodiment, the multimeric protein comprises a mixture of multimeric proteins of different sizes, having a range of numbers of polypeptide monomer units.

Each polypeptide monomer unit of the invention comprises two individual polypeptide chains. The two polypeptide chains within a particular polypeptide monomer unit may be the same as one another, or they may be different from one another. In one embodiment, the two polypeptide chains are the same as one another.

Similarly, the polypeptide monomer units within a particular multimeric protein may be the same as one another, or they may be different from one another. In one embodiment, the polypeptide monomer units are the same as one another.

In one embodiment, a polypeptide chain of a polypeptide monomer unit comprises an amino acid sequence as provided in FIG. 1, optionally with an alternative hinge sequence.

Accordingly in one example the present invention also provides a multimeric protein comprising or consisting of two or more, polypeptide monomer units;

wherein each polypeptide monomer unit comprises two identical polypeptide chains each polypeptide chain comprising or consisting of the sequence given in any one of SEQ ID Nos. 24 to 30 and

wherein the polypeptide monomer unit does not comprise an antibody CH1 domain.

In one example where the hinge may be varied from the sequences given in SEQ ID NOS. 24 to 30 the present invention provides a multimeric protein comprising two or more polypeptide monomer units;

wherein each polypeptide monomer unit comprises an antibody Fc-domain comprising two heavy chain Fc-regions,

wherein each heavy chain Fc-region comprises or consists of the sequence given in amino acids 6 to 222 of any one of SEQ ID NOs 24 to 27 and 30 or the sequence given in amino acids 6 to 333 of SEQ ID NOs 28 or 29 and wherein the polypeptide monomer unit does not comprise an antibody CH1 domain.

Typically, each heavy chain Fc-region comprises a hinge sequence at the N-terminus.

The multimeric proteins of the present invention may comprise one or more mutations that alter the functional properties of the proteins, for example, binding to Fc-receptors such as FcRn or leukocyte receptors, binding to complement, modified disulphide bond architecture or altered glycosylation patterns, as described herein below. It will be appreciated that any of these mutations may be combined in any suitable manner to achieve the desired functional properties, and/or combined with other mutations to alter the functional properties of the proteins.

The multimeric protein of the invention may show altered binding to one or more Fc-receptors (FcR's) in comparison with the corresponding polypeptide monomer unit and/or native immunoglobulin. The binding to any particular Fc-receptor may be increased or decreased. In one embodiment, the multimeric protein of the invention comprises one or more mutations which alter its Fc-receptor binding profile.

The term “mutation” as used herein may include substitution, addition or deletion of one or more amino acids.

Human cells can express a number of membrane bound FcR's selected from FcαR, FcεR, FcγR, FcRn and glycan receptors. Some cells are also capable of expressing soluble (ectodomain) FcR (Fridman et al., (1993) J Leukocyte Biology 54: 504-512 for review). FcγR can be further divided by affinity of IgG binding (high/low) and biological effect (activating/inhibiting). Human FcγRI is widely considered to be the sole ‘high affinity’ receptor whilst all of the others are considered as medium to low. FcγRIIb is the sole receptor with ‘inhibitory’ functionality by virtue of its intracellular ITIM motif whilst all of the others are considered as ‘activating’ by virtue of ITAM motifs or pairing with the common FcγR—γchain. FcγRIIIb is also unique in that although activatory it associates with the cell via a GPI anchor. In total, humans express six ‘standard’ FcγR: FcγRI, FcγRIIa, FcγRIIb, FcγRIIc, FcγRIIIa FcγRIIIb. In addition to these sequences there are a large number of sequence or allotypic variants spread across these families. Some of these have been found to have important functional consequence and so are sometimes considered to be receptor sub-types of their own. Examples include FcγRIIa^{H134R}, FcγRIIb^{I190T}, FcγRIIIa^{F158V} and FcγRIIIb^{NA1}, FcγRIIIb^{NA2} FcγRIII^{SH}. Each receptor sequence has been shown to have different affinities for the 4 sub-classes of IgG: IgG1, IgG2, IgG3 and IgG4 (Bruhns Blood (1993) Vol 113, p3716-3725). Other species have somewhat different numbers and functionality of FcγR, with the mouse system being the best studied to date and comprising of 4 FcγR, FcγRI FcγRIIb FcγRIII FcγRIV (Bruhns, Blood (2012) Vol 119, p5640-5649). Human FcγRI on cells is normally considered to be ‘occupied’ by monomeric IgG in normal serum conditions due to its affinity for IgG1/IgG3/IgG4 (˜10⁻⁸ M) and the concentration of these IgG in serum (˜10 mg/ml). Hence cells bearing FcγRI on their surface are considered to be capable for ‘screening’ or ‘sampling’ of their antigenic environment vicariously through the bound polyspecific IgG. The other receptors having lower affinities for IgG sub-classes (in the range of (˜10⁻⁵−10⁻⁷ M) are normally considered to be ‘unoccupied’. The low affinity receptors are hence inherently sensitive to the detection of and activation by antibody involved immune complexes. The increased Fc density in an antibody immune complex results in increased functional affinity of binding ‘avidity’ to low affinity FcγR. This has been demonstrated in vitro using a number of methods (Shields R. L. et al, J Biol Chem, Vol 276(9), p6591-6604, 2001; Lux et al., J Immunol (2013) Vol 190, p4315-4323). It has also been implicated as being one of the primary modes of action in the use of anti-RhD to treat ITP in humans (Crow Transfusion Medicine Reviews (2008) Vol 22, p103-116).

Many cell types express multiple types of FcγR and so binding of IgG or antibody immune complex to cells bearing FcγR can have multiple and complex outcomes depending upon the biological context. Most simply, cells can either receive an activatory, inhibitory or mixed signal. This can result in events such as phagocytosis (e.g. macrophages and neutrophils), antigen processing (e.g. dendritic cells), reduced IgG production (e.g. B-cells) or degranulation (e.g. neutrophils, mast cells). There are data to support that the inhibitory signal from FcγRIIb can dominate that of activatory signals (Proulx Clinical Immunology (2010) 135:422-429.

FcRn has a crucial role in maintaining the long half-life of IgG in the serum of adults and children. The receptor binds IgG in acidified vesicles (pH<6.5) protecting the IgG molecule from degradation, and then releasing it at the higher pH of 7.4 in blood.

FcRn is unlike leukocyte Fc receptors, and instead, has structural similarity to MHC class I molecules. It is a heterodimer composed of a β₂-microglobulin chain, non-covalently attached to a membrane-bound chain that includes three extracellular domains. One of these domains, including a carbohydrate chain, together with β₂-microglobulin interacts with a site between the CH2 and CH3 domains of Fc. The interaction includes salt bridges made to histidine residues on IgG that are positively charged at pH<6.5. At higher pH, the His residues lose their positive charges, the FcRn-IgG interaction is weakened and IgG dissociates.

In one embodiment, the multimeric protein of the invention binds to human FcRn.

In one embodiment, the multimeric protein has a histidine residue at position 310, and preferably also at position 435. These histidine residues are important for human FcRn binding. In one embodiment, the histidine residues at positions 310 and 435 are native residues, i.e. positions 310 and 435 are not mutated. Alternatively, one or both of these histidine residues may be present as a result of a mutation.

In one embodiment the present invention provides a multimeric protein comprising two or more polypeptide monomer units;

wherein each polypeptide monomer unit comprises an antibody Fc-domain comprising two heavy chain Fc-regions,

wherein each heavy chain Fc-region comprises a cysteine residue at position 309 which causes the monomer units to assemble into a multimer, and a histidine residue at position 310, and

wherein each polypeptide monomer unit does not comprise a CH1 domain or a tailpiece.

The multimeric protein of the invention may comprise one or more mutations which alter its binding to FcRn. The altered binding may be increased binding or decreased binding.

In one embodiment, the multimeric protein comprises one or more mutations such that it binds to FcRn with greater affinity and avidity than the corresponding native immunoglobulin.

In one embodiment, the Fc domain is mutated by substituting the threonine residue at position 250 with a glutamine residue (T250Q).

In one embodiment, the Fc domain is mutated by substituting the methionine residue at position 252 with a tyrosine residue (M252Y)

In one embodiment, the Fc domain is mutated by substituting the serine residue at position 254 with a threonine residue (S254T).

In one embodiment, the Fc domain is mutated by substituting the threonine residue at position 256 with a glutamic acid residue (T256E).

In one embodiment, the Fc domain is mutated by substituting the threonine residue at position 307 with an alanine residue (T307A).

In one embodiment, the Fc domain is mutated by substituting the threonine residue at position 307 with a proline residue (T307P).

In one embodiment, the Fc domain is mutated by substituting the valine residue at position 308 with a cysteine residue (V308C).

In one embodiment, the Fc domain is mutated by substituting the valine residue at position 308 with a phenylalanine residue (V308F).

In one embodiment, the Fc domain is mutated by substituting the valine residue at position 308 with a proline residue (V308P).

In one embodiment, the Fc domain is mutated by substituting the glutamine residue at position 311 with an alanine residue (Q311A).

In one embodiment, the Fc domain is mutated by substituting the glutamine residue at position 311 with an arginine residue (Q311R).

In one embodiment, the Fc domain is mutated by substituting the methionine residue at position 428 with a leucine residue (M428L).

In one embodiment, the Fc domain is mutated by substituting the histidine residue at position 433 with a lysine residue (H433K).

In one embodiment, the Fc domain is mutated by substituting the asparagine residue at position 434 with a phenylalanine residue (N434F).

In one embodiment, the Fc domain is mutated by substituting the asparagine residue at position 434 with a tyrosine residue (N434Y).

In one embodiment, the Fc domain is mutated by substituting the methionine residue at position 252 with a tyrosine residue, the serine residue at position 254 with a threonine residue, and the threonine residue at position 256 with a glutamic acid residue (M252Y/S254T/T256E).

In one embodiment, the Fc domain is mutated by substituting the valine residue at position 308 with a proline residue and the asparagine residue at position 434 with a tyrosine residue (V308P/N434Y).

In one embodiment, the Fc domain is mutated by substituting the methionine residue at position 252 with a tyrosine residue, the serine residue at position 254 with a threonine residue, the threonine residue at position 256 with a glutamic acid residue, the histidine residue at position 433 with a lysine residue and the asparagine residue at position 434 with a phenylalanine residue (M252Y/S254T/T256E/H433K/N434F).

It will be appreciated that any of the mutations listed above may be combined to alter FcRn binding.

In one embodiment, the multimeric protein comprises one or more mutations such that it binds to FcRn with lower affinity and avidity than the corresponding native immunoglobulin.

In one embodiment, the Fc domain comprises any amino acid residue other than histidine at position 310 and/or position 435.

The multimeric protein of the invention may comprise one or more mutations which increase its binding to FcγRIIb. FcγRIIb is the only inhibitory receptor in humans and the only Fc receptor found on B cells. B cells and their pathogenic antibodies lie at the heart of many immune diseases, and thus the multimeric proteins may provide improved therapies for these diseases.

In one embodiment, the Fc domain is mutated by substituting the proline residue at position 238 with an aspartic acid residue (P238D).

In one embodiment, the Fc domain is mutated by substituting the glutamic acid residue at position 258 with an alanine residue (E258A).

In one embodiment, the Fc domain is mutated by substituting the serine residue at position 267 with an alanine residue (S267A).

In one embodiment, the Fc domain is mutated by substituting the serine residue at position 267 with a glutamic acid residue (S267E).

In one embodiment, the Fc domain is mutated by substituting the leucine residue at position 328 with a phenylalanine residue (L328F).

In one embodiment, the Fc domain is mutated by substituting the glutamic acid residue at position 258 with an alanine residue and the serine residue at position 267 with an alanine residue (E258A/S267A).

In one embodiment, the Fc domain is mutated by substituting the serine residue at position 267 with a glutamic acid residue and the leucine residue at position 328 with a phenylalanine residue (S267E/L328F).

It will be appreciated that any of the mutations listed above may be combined to increase FcγRIIb binding.

In one embodiment of the invention we provide multimeric proteins which display decreased binding to FcγR. Decreased binding to FcγR may provide improved therapies for use in the treatment of immune diseases involving pathogenic antibodies.

In one embodiment the multimeric protein of the present invention comprises one or more mutations that decrease its binding to FcγR.

In one embodiment, a mutation that decreases binding to FcγR is used in a multimeric protein of the invention which comprises an Fc-domain derived from IgG1.

In one embodiment, the Fc domain is mutated by substituting the leucine residue at position 234 with an alanine residue (L234A).

In one embodiment, the Fc domain is mutated by substituting the leucine residue at position 235 with an alanine residue (L235A).

In one embodiment, the Fc-domain is mutated by substituting the glycine residue at position 236 with an arginine residue (G236R).

In one embodiment, the Fc domain is mutated by substituting the asparagine residue at position 297 with an alanine residue (N297A) or a glutamine residue (N297Q).

In one embodiment, the Fc domain is mutated by substituting the serine residue at position 298 with an alanine residue (S298A).

In one embodiment, the Fc domain is mutated by substituting the leucine residue at position 328 with an arginine residue (L328R).

In one embodiment, the Fc-domain is mutated by substituting the leucine residue at position 234 with an alanine residue and the leucine residue at position 235 with an alanine residue (L234A/L235A).

In one embodiment, the Fc-domain is mutated by substituting the phenylalanine residue at position 234 with an alanine residue and the leucine residue at position 235 with an alanine residue (F234A/L235A).

In one embodiment, the Fc domain is mutated by substituting the glycine residue at position 236 with an arginine residue and the leucine residue at position 328 with an arginine residue (G236R/L328R).

It will be appreciated that any of the mutations listed above may be combined to decrease FcγR binding.

In one embodiment the multimeric protein of the present invention comprises one or more mutations that decrease its binding to FcγRIIIa without affecting its binding to FcγRII.

In one embodiment, the Fc domain is mutated by substituting the serine residue at position 239 with an alanine residue (S239A).

In one embodiment, the Fc domain is mutated by substituting the glutamic acid residue at position 269 with an alanine residue (E269A).

In one embodiment, the Fc domain is mutated by substituting the glutamic acid residue at position 293 with an alanine residue (E293A).

In one embodiment, the Fc domain is mutated by substituting the tyrosine residue at position 296 with a phenylalanine residue (Y296F).

In one embodiment, the Fc domain is mutated by substituting the valine residue at position 303 with an alanine residue (V303A).

In one embodiment, the Fc domain is mutated by substituting the alanine residue at position 327 with a glycine residue (A327G).

In one embodiment, the Fc domain is mutated by substituting the lysine residue at position 338 with an alanine residue (K338A).

In one embodiment, the Fc domain is mutated by substituting the aspartic acid residue at position 376 with an alanine residue (D376A).

It will be appreciated that any of the mutations listed above may be combined to decrease FcγRIIIa binding.

The multimeric protein of the invention may comprise one or more mutations that alter its binding to complement. Altered complement binding may be increased binding or decreased binding.

In one embodiment the protein comprises one or more mutations which decrease its binding to C1q. Initiation of the classical complement pathway starts with binding of hexameric C1q protein to the CH2 domain of antigen bound IgG and IgM. The multimeric proteins of the invention do not possess antigen binding sites, and so would not be expected to show significant binding to C1q. However, the presence of one or more mutations that decrease C1q binding will ensure that they do not activate complement in the absence of antigen engagement, so providing improved therapies with greater safety.

Thus in one embodiment the multimeric protein of the invention comprises one or more mutations to decrease its binding to C1q.

In one embodiment, the Fc domain is mutated by substituting the leucine residue at position 234 with an alanine residue (L234A).

In one embodiment, the Fc domain is mutated by substituting the leucine residue at position 235 with an alanine residue (L235A).

In one embodiment, the Fc domain is mutated by substituting the leucine residue at position 235 with a glutamic acid residue (L235E).

In one embodiment, the Fc domain is mutated by substituting the glycine residue at position 237 with an alanine residue (G237A).

In one embodiment, the Fc domain is mutated by substituting the lysine residue at position 322 with an alanine residue (K322A).

In one embodiment, the Fc domain is mutated by substituting the proline residue at position 331 with an alanine residue (P331A).

In one embodiment, the Fc domain is mutated by substituting the proline residue at position 331 with a serine residue (P331S).

In one embodiment, the multimeric protein comprises an Fc domain derived from IgG4. IgG4 has a naturally lower complement activation profile than IgG1, but also weaker binding of FcγR. Thus, in one embodiment, the multimeric protein comprising IgG4 also comprises one or more mutations that increase FcγR binding.

It will be appreciated that any of the mutations listed above may be combined to reduce C1q binding.

The antibody Fc-domain of the invention comprises one or more mutations to create and/or remove a cysteine residue. Cysteine residues have an important role in the spontaneous assembly of the multimeric protein, by forming disulphide bridges between individual pairs of polypeptide monomer units. Thus, by altering the number and/or position of cysteine residues, it is possible to modify the structure of the multimeric protein to produce a protein with improved therapeutic properties.

The multimeric protein of the present invention comprises a cysteine residue at position 309. In one embodiment, the cysteine residue at position 309 is created by a mutation, e.g. for an Fc-domain derived from IgG1, the leucine residue at position 309 is substituted with a cysteine residue (L309C), for an Fc-domain derived from IgG2, the valine residue at position 309 is substituted with a cysteine residue (V309C).

In one embodiment, the antibody Fc-domain is mutated by substituting the valine residue at position 308 with a cysteine residue (V308C).

In one embodiment, two disulphide bonds in the hinge region are removed by mutating a core hinge sequence CPPC to SPPS.

In one embodiment of the invention we provide multimeric proteins with improved manufacturability comprising fewer glycosylation sites. These proteins have less complex post translational glycosylation patterns and are thus simpler and less expensive to manufacture.

In one embodiment a glycosylation site in the CH2 domain is removed by substituting the asparagine residue at position 297 with an alanine residue (N297A) or a glutamine residue (N297Q). In addition to improved manufacturability, these aglycosyl mutants also reduce FcγR binding as described herein above.

It will be appreciated that any of the mutations listed above may be combined.

The present invention also provides an isolated DNA sequence encoding a polypeptide chain of a polypeptide monomer unit of the present invention, or a component part thereof. The DNA sequence may comprise synthetic DNA, for instance produced by chemical processing, cDNA, genomic DNA or any combination thereof.

DNA sequences which encode a polypeptide chain of a polypeptide monomer unit of the present invention can be obtained by methods well known to those skilled in the art. For example, DNA sequences coding for part or all of a polypeptide chain of a polypeptide monomer unit may be synthesised as desired from the determined DNA sequences or on the basis of the corresponding amino acid sequences.

Standard techniques of molecular biology may be used to prepare DNA sequences coding for a polypeptide chain of a polypeptide monomer unit of the present invention. Desired DNA sequences may be synthesised completely or in part using oligonucleotide synthesis techniques. Site-directed mutagenesis and polymerase chain reaction (PCR) techniques may be used as appropriate.

The present invention also relates to a cloning or expression vector comprising one or more DNA sequences of the present invention. Accordingly, provided is a cloning or expression vector comprising one or more DNA sequences encoding a polypeptide chain of a polypeptide monomer unit of the present invention, or a component part thereof.

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

Also provided is a host cell comprising one or more cloning or expression vectors comprising one or more DNA sequences encoding a multimeric protein of the present invention. Any suitable host cell/vector system may be used for expression of the DNA sequences encoding the multimeric protein of the present invention. Bacterial, for example E. coli, and other microbial systems such as Saccharomyces or Pichia may be used or eukaryotic, for example mammalian, host cell expression systems may also be used. Suitable mammalian host cells include CHO cells. Suitable types of chinese hamster ovary (CHO cells) for use in the present invention may include CHO and CHO-K1 cells, including dhfr- CHO cells, such as CHO-DG44 cells and CHO-DX611 cells, which may be used with a DHFR selectable marker, or CHOK1-sv cells which may be used with a glutamine synthetase selectable marker. Other suitable host cells include NSO cells and HEK cells.

The present invention also provides a process for the production of a multimeric protein according to the present invention, comprising culturing a host cell containing a vector of the present invention under conditions suitable for expression of the protein and assembly into multimers, and isolating and optionally purifying the multimeric protein.

The multimeric proteins of the present invention are expressed at good levels from host cells. Thus the properties of the multimeric protein are conducive to commercial processing.

The multimeric proteins of the present invention may be made using any suitable method. In one embodiment, the multimeric protein of the invention may be produced under conditions which minimise aggregation. In one example, aggregation may be minimised by the addition of preservative to the culture media, culture supernatant, or purification media. Examples of suitable preservatives include thiol capping agents such as N-ethyl maleimide, iodoacetic acid, β-mercaptoethanol, β-mercaptoethylamine, glutathione, or cysteine. Other examples include disulphide inhibiting agents such as ethylenediaminetetraacetic acid (EDTA), ethyleneglycoltetraacetic acid (EGTA), or acidification to below pH 6.0.

In one embodiment there is provided a process for purifying a multimeric protein of the present invention comprising the steps: performing anion exchange chromatography in non-binding mode such that the impurities are retained on the column and the multimeric protein is eluted.

In one embodiment the purification employs affinity capture on an FcRn, FcγR or C-reactive protein column.

In one embodiment the purification employs protein A.

Suitable ion exchange resins for use in the process include Q.FF resin (supplied by GE-Healthcare). The step may, for example be performed at a pH about 8. The process may further comprise an initial capture step employing cation exchange chromatography, performed for example at a pH of about 4 to 5, such as 4.5. The cation exchange chromatography may, for example employ a resin such as CaptoS resin or SP sepharose FF (supplied by GE-Healthcare). The multimeric protein can then be eluted from the resin employing an ionic salt solution such as sodium chloride, for example at a concentration of 200 mM.

The chromatography step or steps may include one or more washing steps, as appropriate.

The purification process may also comprise one or more filtration steps, such as a diafiltration step.

Multimers which have the required number of polypeptide monomer units can be separated according to molecular size, for example by size exclusion chromatography.

Thus in one embodiment there is provided a purified multimeric protein according to the invention, in substantially purified from, in particular free or substantially free of endotoxin and/or host cell protein or DNA.

Purified form as used supra is intended to refer to at least 90% purity, such as 91, 92, 93, 94, 95, 96, 97, 98, 99% w/w or more pure.

Substantially free of endotoxin is generally intended to refer to an endotoxin content of 1 EU per mg antibody product or less such as 0.5 or 0.1 EU per mg product.

Substantially free of host cell protein or DNA is generally intended to refer to host cell protein and/or DNA content 400 μg per mg of protein product or less such as 100 μg per mg or less, in particular 20 μg per mg, as appropriate.

As the multimeric proteins of the present invention are useful in the treatment and/or prophylaxis of a pathological condition, the present invention also provides a pharmaceutical or diagnostic composition comprising a multimeric protein of the present invention in combination with one or more of a pharmaceutically acceptable excipient, diluent or carrier. Accordingly, provided is the use of a protein of the invention for the manufacture of a medicament. The composition will usually be supplied as part of a sterile, pharmaceutical composition that will normally include a pharmaceutically acceptable carrier. A pharmaceutical composition of the present invention may additionally comprise a pharmaceutically acceptable excipient.

The present invention also provides a process for preparation of a pharmaceutical or diagnostic composition comprising adding and mixing the multimeric protein of the present invention together with one or more of a pharmaceutically acceptable excipient, diluent or carrier.

The multimeric protein may be the sole active ingredient in the pharmaceutical or diagnostic composition or may be accompanied by other active ingredients including other antibody ingredients or non-antibody ingredients such as steroids or other drug molecules.

The pharmaceutical compositions suitably comprise a therapeutically effective amount of the multimeric protein of the invention. The term “therapeutically effective amount” as used herein refers to an amount of a therapeutic agent needed to treat, ameliorate or prevent a targeted disease or condition, or to exhibit a detectable therapeutic or preventative effect. For any medicine, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. This amount can be determined by routine experimentation and is within the judgement of the clinician. Generally, a therapeutically effective amount will be from 0.01 mg/kg to 500 mg/kg, for example 0.1 mg/kg to 200 mg/kg, such as 100 mg/kg. Pharmaceutical compositions may be conveniently presented in unit dose forms containing a predetermined amount of an active agent of the invention per dose.

Therapeutic doses of the multimeric protein according to the present disclosure show no apparent toxicology effects in vivo.

In one embodiment of a multimeric protein according to the invention a single dose may provide up to a 90% reduction in circulating IgG levels.

Compositions may be administered individually to a patient or may be administered in combination (e.g. simultaneously, sequentially or separately) with other agents, drugs or hormones.

In one embodiment the multimeric proteins according to the present disclosure are employed with an immunosuppressant therapy, such as a steroid, in particular prednisone.

In one embodiment the multimeric proteins according to the present disclosure are employed with Rituximab or other B cell therapies.

In one embodiment the multimeric proteins according to the present disclosure are employed with any B cell or T cell modulating agent or immunomodulator. Examples include methotrexate, mycophenylate and azathioprine.

The dose at which the multimeric protein of the present invention is administered depends on the nature of the condition to be treated, the extent of the disease present and on whether the multimeric protein is being used prophylactically or to treat an existing condition.

The frequency of dose will depend on the half-life of the multimeric protein and the duration of its effect. If the multimeric protein has a short half-life (e.g. 2 to 10 hours) it may be necessary to give one or more doses per day. Alternatively, if the multimeric protein has a long half-life (e.g. 2 to 15 days) and/or long lasting pharmacodynamic effects it may only be necessary to give a dosage once per day, once per week or even once every 1 or 2 months.

In one embodiment the dose is delivered bi-weekly, i.e. twice a month.

Half-life as employed herein is intended to refer to the duration of the molecule in circulation, for example in serum/plasma.

Pharmacodynamics as employed herein refers to the profile and in particular duration of the biological action of the multimeric protein according the present disclosure.

The pharmaceutically acceptable carrier should not itself induce the production of antibodies harmful to the individual receiving the composition and should not be toxic. Suitable carriers may be large, slowly metabolised macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the patient.

Suitable forms for administration include forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilising and/or dispersing agents. The protein may be in the form of nanoparticles. Alternatively, the antibody molecule may be in dry form, for reconstitution before use with an appropriate sterile liquid.

Once formulated, the compositions of the invention can be administered directly to the subject. The subjects to be treated can be animals. However, in one or more embodiments the compositions are adapted for administration to human subjects.

Suitably in formulations according to the present disclosure, the pH of the final formulation is not similar to the value of the isoelectric point of the multimeric protein, for example if the pl of the protein is in the range 8-9 or above then a formulation pH of 7 may be appropriate. Whilst not wishing to be bound by theory it is thought that this may ultimately provide a final formulation with improved stability, for example the multimeric protein remains in solution.

In one example the pharmaceutical formulation at a pH in the range of 4.0 to 7.0 comprises: 1 to 200 mg/mL of an protein molecule according to the present disclosure, 1 to 100 mM of a buffer, 0.001 to 1% of a surfactant, a) 10 to 500 mM of a stabiliser, b) 10 to 500 mM of a stabiliser and 5 to 500 mM of a tonicity agent, or c) 5 to 500 mM of a tonicity agent.

The pharmaceutical compositions of this invention may be administered by any number of routes including, but not limited to, oral, intravenous, intramuscular, intra-arterial, intramedullary, intrathecal, intraventricular, transdermal, transcutaneous (for example, see WO98/20734), subcutaneous, intraperitoneal, intranasal, enteral, topical, sublingual, intravaginal or rectal routes. Hyposprays may also be used to administer the pharmaceutical compositions of the invention. Typically, the therapeutic compositions may be prepared as injectables, either as liquid solutions or suspensions. Solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule

It will be appreciated that the active ingredient in the composition will be a protein molecule. As such, it will be susceptible to degradation in the gastrointestinal tract. Thus, if the composition is to be administered by a route using the gastrointestinal tract, the composition will need to contain agents which protect the protein from degradation but which release the protein once it has been absorbed from the gastrointestinal tract.

A thorough discussion of pharmaceutically acceptable carriers is available in Remington's Pharmaceutical Sciences (Mack Publishing Company, N.J. 1991). In one embodiment the formulation is provided as a formulation for topical administrations including inhalation.

Suitable inhalable preparations include inhalable powders, metering aerosols containing propellant gases or inhalable solutions free from propellant gases. Inhalable powders according to the disclosure containing the active substance may consist solely of the above mentioned active substances or of a mixture of the abovementioned active substances with physiologically acceptable excipient. These inhalable powders may include monosaccharides (e.g. glucose or arabinose), disaccharides (e.g. lactose, saccharose, maltose), oligo- and polysaccharides (e.g. dextranes), polyalcohols (e.g. sorbitol, mannitol, xylitol), salts (e.g. sodium chloride, calcium carbonate) or mixtures of these with one another. Mono- or disaccharides are suitably used, the use of lactose or glucose, particularly but not exclusively in the form of their hydrates.

Particles for deposition in the lung require a particle size less than 10 microns, such as 1-9 microns for example from 1 to 5 μm. The particle size of the active ingredient (such as the antibody or fragment) is of primary importance.

The propellant gases which can be used to prepare the inhalable aerosols are known in the art. Suitable propellant gases are selected from among hydrocarbons such as n-propane, n-butane or isobutane and halohydrocarbons such as chlorinated and/or fluorinated derivatives of methane, ethane, propane, butane, cyclopropane or cyclobutane. The abovementioned propellant gases may be used on their own or in mixtures thereof.

Particularly suitable propellant gases are halogenated alkane derivatives selected from among TG 11, TG 12, TG 134a and TG227. Of the abovementioned halogenated hydrocarbons, TG134a (1,1,1,2-tetrafluoroethane) and TG227 (1,1,1,2,3,3,3-heptafluoropropane) and mixtures thereof are particularly suitable.

The propellant-gas-containing inhalable aerosols may also contain other ingredients such as cosolvents, stabilisers, surface-active agents (surfactants), antioxidants, lubricants and means for adjusting the pH. All these ingredients are known in the art.

The propellant-gas-containing inhalable aerosols according to the invention may contain up to 5% by weight of active substance. Aerosols according to the invention contain, for example, 0.002 to 5% by weight, 0.01 to 3% by weight, 0.015 to 2% by weight, 0.1 to 2% by weight, 0.5 to 2% by weight or 0.5 to 1% by weight of active ingredient.

Alternatively topical administrations to the lung may also be by administration of a liquid solution or suspension formulation, for example employing a device such as a nebulizer, for example, a nebulizer connected to a compressor (e.g., the Pari LC-Jet Plus(R) nebulizer connected to a Pad Master(R) compressor manufactured by Pari Respiratory Equipment, Inc., Richmond, Va.).

The protein of the invention can be delivered dispersed in a solvent, e.g., in the form of a solution or a suspension. It can be suspended in an appropriate physiological solution, e.g., saline or other pharmacologically acceptable solvent or a buffered solution. Buffered solutions known in the art may contain 0.05 mg to 0.15 mg disodium edetate, 8.0 mg to 9.0 mg NaCl, 0.15 mg to 0.25 mg polysorbate, 0.25 mg to 0.30 mg anhydrous citric acid, and 0.45 mg to 0.55 mg sodium citrate per 1 ml of water so as to achieve a pH of about 4.0 to 5.0. A suspension can employ, for example, lyophilised protein.

The therapeutic suspensions or solution formulations can also contain one or more excipients. Excipients are well known in the art and include buffers (e.g., citrate buffer, phosphate buffer, acetate buffer and bicarbonate buffer), amino acids, urea, alcohols, ascorbic acid, phospholipids, proteins (e.g., serum albumin), EDTA, sodium chloride, liposomes, mannitol, sorbitol, and glycerol. Solutions or suspensions can be encapsulated in liposomes or biodegradable microspheres. The formulation will generally be provided in a substantially sterile form employing sterile manufacture processes.

This may include production and sterilization by filtration of the buffered solvent/solution used for the formulation, aseptic suspension of the protein in the sterile buffered solvent solution, and dispensing of the formulation into sterile receptacles by methods familiar to those of ordinary skill in the art.

Nebulizable formulation according to the present disclosure may be provided, for example, as single dose units (e.g., sealed plastic containers or vials) packed in foil envelopes. Each vial contains a unit dose in a volume, e.g., 2 ml, of solvent/solution buffer.

The multimeric proteins disclosed herein may be suitable for delivery via nebulisation.

It is also envisaged that the proteins of the present invention may be administered by use of gene therapy. In order to achieve this, DNA sequences encoding the polypeptide chains of the protein molecule under the control of appropriate DNA components are introduced into a patient such that the polypeptide chains are expressed from the DNA sequences and assembled in situ.

In one embodiment we provide the multimeric protein of the invention for use in therapy.

In one embodiment we provide the multimeric protein of the invention for use in the treatment of immune disorders.

In one embodiment we provide the multimeric protein of the invention for use in the treatment of cancer.

In one embodiment we provide the multimeric protein of the invention for use as a vaccine.

In one embodiment we provide the use of the multimeric protein of the invention for the preparation of a medicament for the treatment of immune disorders.

In one embodiment we provide the use of the multimeric protein of the invention for the preparation of a medicament for the treatment of cancer.

In one embodiment we provide the use of the multimeric protein of the invention for the preparation of a vaccine.

Examples of immune disorders which may be treated using the multimeric protein of the invention include immune thrombocytopenia (ITP), chronic inflammatory demyelinating polyneuropathy (CIDP), Kawasaki disease and Guillain-Barré syndrome (GBS).

The present invention also provides a multimeric protein (or compositions comprising same) for use in the control of autoimmune diseases, for example Acute Disseminated Encephalomyelitis (ADEM), Acute necrotizing hemorrhagic leukoencephalitis, Addison's disease, Agammaglobulinemia, Alopecia areata, Amyloidosis, ANCA-associated vasculitis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency , Autoimmune inner ear disease (AIED), Autoimmune myocarditis, Autoimmune pancreatitis, Autoimmune retinopathy, Autoimmune thrombocytopenic purpura (ATP), Autoimmune thyroid disease,

Autoimmune urticarial, Axonal & nal neuropathies, Balo disease, Behcet's disease, Bullous pemphigoid, Cardiomyopathy, Castleman disease, Celiac disease, Chagas disease, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Dilated cardiomyopathy, Discoid lupus, Dressler's syndrome, Endometriosis, Eosinophilic angiocentric fibrosis, Eosinophilic fasciitis, Erythema nodosum, Experimental allergic encephalomyelitis, Evans syndrome, Fibrosing alveolitis, Giant cell arteritis (temporal arteritis), Glomerulonephritis, Goodpasture's syndrome, Granulomatosis with Polyangiitis (GPA) see Wegener's, Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic hypocomplementemic tubulointestitial nephritis, Idiopathic thrombocytopenic purpura (ITP), IgA nephropathy, IgG4-related disease, IgG4-related sclerosing disease, lmmunoregulatory lipoproteins, Inflammatory aortic aneurysm, Inflammatory pseudotumour, Inclusion body myositis, Insulin-dependent diabetes (type1), Interstitial cystitis, Juvenile arthritis, Juvenile diabetes, Kawasaki syndrome, Kuttner's tumour, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Mediastinal fibrosis, Meniere's disease, Microscopic polyangiitis, Mikulicz's syndrome, Mixed connective tissue disease (MCTD), Mooren's ulcer, Mucha-Habermann disease, Multifocal fibrosclerosis, Multiple sclerosis, Myasthenia gravis, Myositis, Narcolepsy, Neuromyelitis optica (Devic's), Neutropenia, Ocular cicatricial pemphigoid, Optic neuritis, Ormond's disease (retroperitoneal fibrosis), Palindromic rheumatism, PANDAS (Pediatric Autoimmune Neuropsychiatric Disorders Associated with Streptococcus), Paraneoplastic cerebellar degeneration, Paraproteinemic polyneuropathies, Paroxysmal nocturnal hemoglobinuria (PNH), Parry Romberg syndrome, Parsonnage-Turner syndrome, Pars planitis (peripheral uveitis), Pemphigus vulgaris, Periaortitis, Periarteritis, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatic, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reflex sympathetic dystrophy, Reiter's syndrome, Relapsing polychondritis, Restless legs syndrome, Retroperitoneal fibrosis (Ormond's disease), Rheumatic fever, Rheumatoid arthritis, Riedel's thyroiditis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombotic, thrombocytopenic purpura (TTP), Tolosa-Hunt syndrome, Transverse myelitis, Ulcerative colitis, Undifferentiated connective tissue disease (UCTD), Uveitis, Vasculitis, Vesiculobullous dermatosis, Vitiligo, Waldenstrom Macroglobulinaemia, Warm idiopathic haemolytic anaemia and Wegener's granulomatosis (now termed Granulomatosis with Polyangiitis (GPA).

In one embodiment the multimeric proteins and fragments according to the disclosure are employed in the treatment or prophylaxis of epilepsy or seizures.

In one embodiment the multimeric proteins and fragments according to the disclosure are employed in the treatment or prophylaxis of multiple sclerosis.

In embodiment the multimeric proteins and fragments of the disclosure are employed in alloimmune disease/indications which includes:
Transplantation donor mismatch due to anti-HLA antibodies
Foetal and neonatal alloimmune thrombocytopenia, FNAIT (or neonatal alloimmune thrombocytopenia, NAITP or NAIT or NAT, or foeto-maternal alloimmune thrombocytopenia, FMAITP or FMAIT).

Additional indications include: rapid clearance of Fc-containing biopharmaceutical drugs from human patients and combination of multimeric protein therapy with other therapies—IVIg, Rituxan, plasmapheresis. For example multimeric protein therapy may be employed following Rituxan therapy.

In embodiment the antibodies and fragments of the disclosure are employed in a neurology disorder such as:
Chronic inflammatory demyelinating polyneuropathy (CIDP)
Guillain-Barré syndrome
Paraproteinemic polyneuropathies
Neuromyelitis optica (NMO, NMO spectrum disorders or NMO spectrum diseases), and
Myasthenia gravis.

In embodiment the antibodies and fragments of the disclosure are employed in a dermatology disorder such as:
Bullous pemphigoid
Pemphigus vulgaris
ANCA-associated vasculitis
Dilated cardiomyopathy

In one embodiment the proteins of the disclosure are employed in an immunology or haematology disorder such as:
Idiopathic thrombocytopenic purpura (ITP)
Thrombotic thrombocytopenic purpura (TTP)
Warm idiopathic haemolytic anaemia
Goodpasture's syndrome
Transplantation donor mismatch due to anti-HLA antibodies

In one embodiment the disorder is selected from Myasthenia Gravis, Neuro- myelitis Optica, CIDP, Guillain-Barré syndrome, Para-proteinemic Poly neuropathy, Refractory Epilepsy, ITP/TTP, Hemolytic Anemia, Goodpasture's Syndrome, ABO mismatch, Lupus nephritis, Renal Vasculitis, Sclero-derma, Fibrosing alveolitis, Dilated cardio-myopathy, Grave's Disease, Type 1 diabetes, Auto-immune diabetes, Pemphigus, Sclero-derma, Lupus, ANCA vasculitis, Dermato-myositis, Sjogren's Disease and Rheumatoid Arthritis.

In one embodiment the disorder is selected from autoimmune polyendocrine syndrome types 1 (APECED or Whitaker's Syndrome) and 2 (Schmidt's Syndrome); alopecia universalis, myasthenic crisis; thyroid crisis; thyroid associated eye disease; thyroid ophthalmopathy, autoimmune diabetes; autoantibody associated encephalitis and/or encephalopathy, pemphigus foliaceus, epidermolysis bullosa, dermatitis herpetiformis, Sydenham's chorea; acute motor axonal neuropathy (AMAN), Miller-Fisher syndrome; multifocal motor neuropathy (MMN), opsoclonus, inflammatory myopathy, Isaac's syndrome (autoimmune neuromyotonia), Paraneoplastic syndromes and Limbic encephalitis.

Examples of cancers which may be treated using the multimeric protein of the invention include colorectal cancer, hepatoma (liver cancer), prostate cancer, pancreatic cancer, breast cancer, ovarian cancer, thyroid cancer, renal cancer, bladder cancer, head and neck cancer or lung cancer.

In one embodiment the cancer is skin cancer, such as melanoma. In one embodiment the cancer is Leukemia. In one embodiment the cancer is glioblastoma, medulloblastoma or neuroblastoma. In one embodiment the cancer is a neuroendocrine cancer. In one embodiment the cancer is Hodgkin's or non-Hodgkins lymphoma.

The multimeric protein according to the present disclosure may be employed in treatment or prophylaxis.

The present invention also provides a method of reducing the concentration of undesired antibodies in an individual comprising the steps of administering to an individual a therapeutically effective dose of a multimeric protein described herein.

The multimeric protein of the present invention may also be used in diagnosis, for example in the in vivo diagnosis and imaging of disease states involving Fc-receptors, such as B-cell related lymphomas.

### FIGURE LEGENDS

FIG. 1: Example amino acid sequences of a polypeptide chain of a polypeptide monomer unit. In each sequence, mutations are shown in bold and underlined. The optional hinge region is underlined. In constructs comprising a CH4 domain from IgM, this region is shown in italics.

FIG. 2: Overlay of the purified fractions obtained for IgG1-Fc-L309C, transiently expressed in CHO cells, after G3000 size-exclusion HPLC. The results demonstrate that the protein assembles into multimers having a range of numbers of monomer units. The graph shows the presence of monomer, dimer, trimer, tetramer, pentamer, and higher than pentamer forms.

M=monomer

Di=dimer

Tri=trimer

Tet=tetramer

Pent+=pentamer and higher than pentamer

FIG. 3: Effect of the multimeric proteins on antibody-dependent phagocytosis of B-cells by human macrophages. The results demonstrate that the multimeric proteins inhibit phagocytosis by the macrophages. The data shows a clear positive correlation between increasing valency, (i.e. multimers with increasing number of monomers), and increasing potency. The potency and maximum levels of inhibition achieved by the multimers is significantly better than human IVIG.

FIG. 4: Stimulation of cytokine release by the multimeric proteins.

### EXAMPLES

### Example 1

### Molecular Biology

Standard molecular biology methods were used including FOR, restriction-ligation cloning, point mutagenesis (Quikchange) and Sanger sequencing. Expression constructs were cloned into expression vectors suitable for both transient and stable expression in CHO cells. An example of a suitable expression vector is pCDNA3 (Invitrogen).

De novo Design of Synthetic Sequences

DNA sequences were designed to contain flanking restriction sites, HindIII and EcoRI, a Kozak sequence, signal peptide and the gene of interest. Sequences were synthesised by DNA 2.0.

Restriction Enzyme Cloning

Synthesised DNA sequences were subcloned into the expression vector using restriction enzymes HindIII and EcoRI.

Mutagenesis

Mutations to the Fc fragment were introduced by site directed mutagenesis. Oligo's were designed to incorporate the desired mutations and purchased from Sigma. FOR reactions were set up to substitute amino acids using the Agilent Quikchange Lightning mutagenesis kits.

Diagrams showing example amino acid sequences of a polypeptide chain of a polypeptide monomer unit are provided in FIG. 1. In each sequence, mutations are shown in bold and underlined. The optional hinge region is underlined. In constructs comprising a CH4 domain from IgM, this region is shown in italics.

### Example 2

### Expression

Small scale expression was performed using ‘transient’ expression of HEK293 or CHO cells transfected using lipofectamine or electroporation. Cultures were grown in shaking flasks or agitated bags in CD-CHO (Lonza) or ProCHO5 (Life Technologies) media at scales ranging from 50-2000 ml for 5-10 days. Cells were removed by centrifugation and culture supernatants were stored at 4° C. until purified. Preservatives were added to some cultures after removal of cells.

The results demonstrated that the multimeric proteins were expressed well.

### Example 3

### Purification and Analysis

Fc-multimers were purified from culture supernatants after checking/adjusting pH to be 6.5, by protein A chromatography with step elution using a pH3.4 buffer. Eluate was immediately neutralised to −pH7.0 using 1 M Tris pH8.5 before storage at 4° C. Analytical size exclusion chromatography was used to separate various multimeric forms of Fc-domains using S200 columns and fraction collection. Fractions were analysed and pooled after G3000 HPLC and reducing and non-reducing SDS-PAGE analysis. Endotoxin was tested using the limulus amoebocyte lysate (LAL) assay and samples used in assays were <1EU/mg.

Purification of the multimeric proteins in the presence of a preservative reduced the tendency to aggregate, producing improved preparations with more uniform structure. Examples of preservatives shown to be effective include thiol capping agents such as N-ethylmaleimide (NEM) and glutathione (GSH), and disulphide inhibiting agents such as ethylenediaminetetraacetic acid (EDTA).

FIG. 2 shows an overlay of the purified fractions obtained for IgG1-Fc-L309C, transiently expressed in CHO cells, after G3000 size-exclusion HPLC. The results demonstrate that the protein assembles into multimers having a range of numbers of monomer units. The graph shows the presence of monomer, dimer, trimer, tetramer, pentamer, and higher than pentamer forms.

### Example 4

### Macrophage Phagocytosis of B Cell Targets

An assay was designed to measure antibody-dependent phagocytosis of B cells by human macrophages. To prepare macrophages, human peripheral blood mononuclear cells (PBMC) were first isolated from fresh blood by density-gradient centrifugation. Monocytes were then selected by incubating the PBMCs for 1 hour at 37° C. in 6-well tissue culture coated plates, followed by removal of non-adherent cells. Adherent monocytes were differentiated into macrophages by 5 day culture in macrophage-colony stimulating factor (MCSF). Human B cells were then prepared from a separate (allogeneic) donor by isolation of PBMC followed by purification of B cells by negative selection using MACS (B cell isolation kit II, Miltenyi Biotech). In some assays, B cells were labelled with carboxyfluorescein succinimidyl ester (CFSE) (Molecular Probes). Differentiated macrophages and B cells were co-cultured at a 1:5 ratio in the presence of anti-CD20 mAb (rituximab) to induce antibody-dependent phagocytosis of the B cells. Multimeric proteins or controls were added at the indicated concentrations and the cells incubated at 37° C. 5% CO₂ for 1-24 hrs. At the end of each time-point, cells were centrifuged and resuspended in FACS buffer at 4° C. to stop further phagocytosis and the B cells surface-stained with anti-CD19 allophycocyanin (APC) before analysis by flow cytometry. Macrophages were distinguished by their auto-fluorescence/side-scatter properties and B cells by their CFSE/CD19 labelling. CFSE-positive macrophages negative for CD19 labelling were assumed to contain engulfed B cells.

FIG. 3 shows that the multimeric proteins inhibit antibody-dependent phagocytosis of B-cells by human macrophages. The data demonstrates a clear positive correlation between increasing valency, (i.e. multimers with increasing number of monomers), and increasing potency. The potency and maximum levels of inhibition achieved by the multimers is significantly better than human IVIG.

Flow cytometry analysis using CFSE stained B-cells confirmed that the mechanism of action is inhibition of macrophage phagocytosis, and not B-cell killing or apoptosis by other means.

### Example 5

### Human Whole Blood Cytokine Release Assay

Fresh blood was collected from donors in lithium heparin vacutainers. The Fc-multimer constructs of interest or controls were serially diluted in sterile PBS to the indicated concentrations. 12.5 μl of Fc-multimer or control was added to the assay plates, followed by 237.5 μl of whole blood. The plate was incubated at 37° C. without CO₂ supplementation for 24 hrs. Plates were centrifuged at 1800 rpm for 5 minutes and the serum removed for cytokine analysis. Cytokine analysis was performed by Meso Scale Discovery cytokine multiplex according to the manufacturer's protocol and read on a Sector Imager 600.

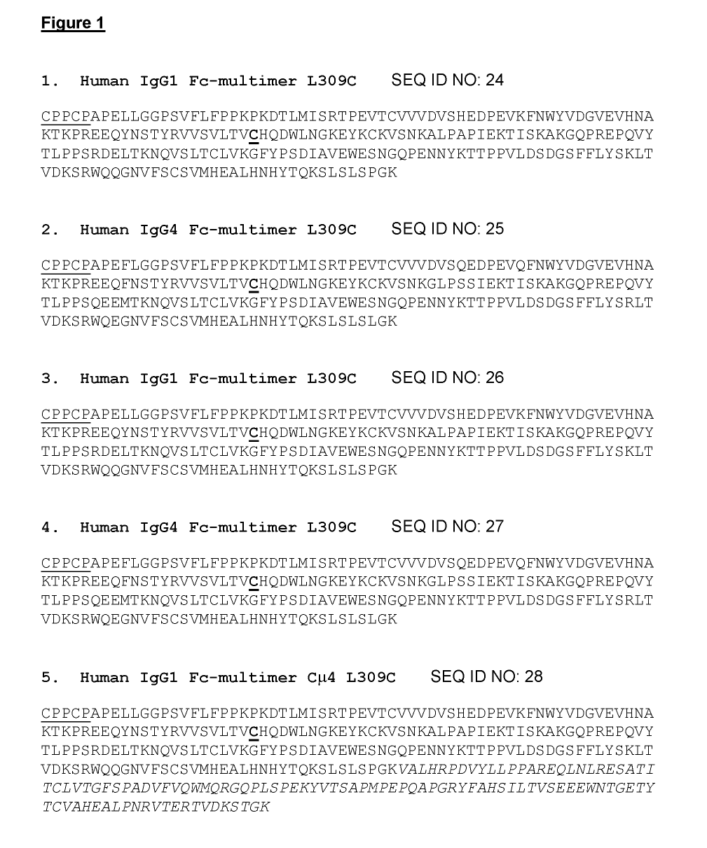

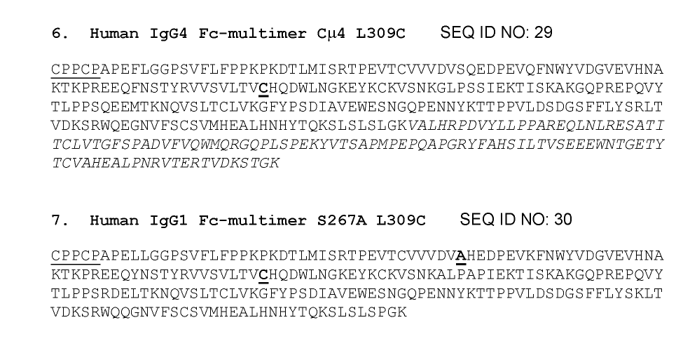

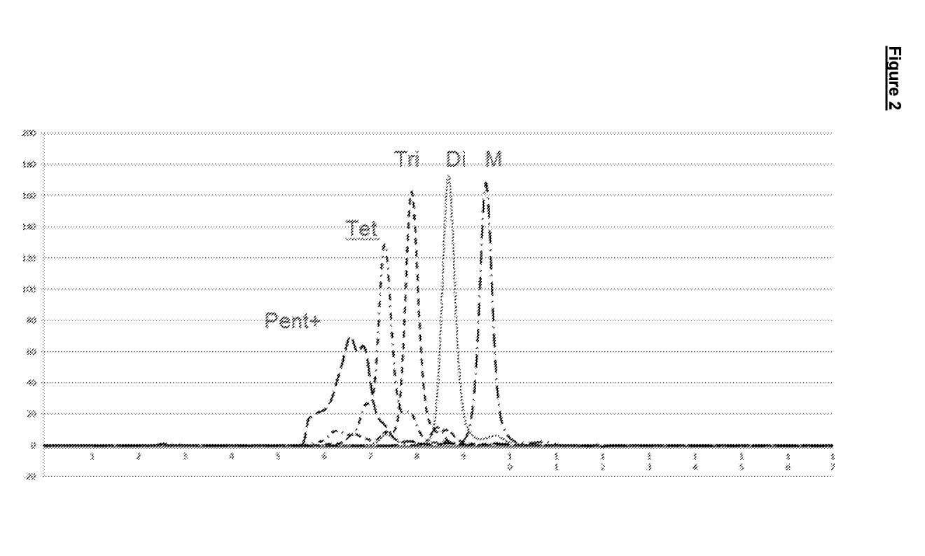

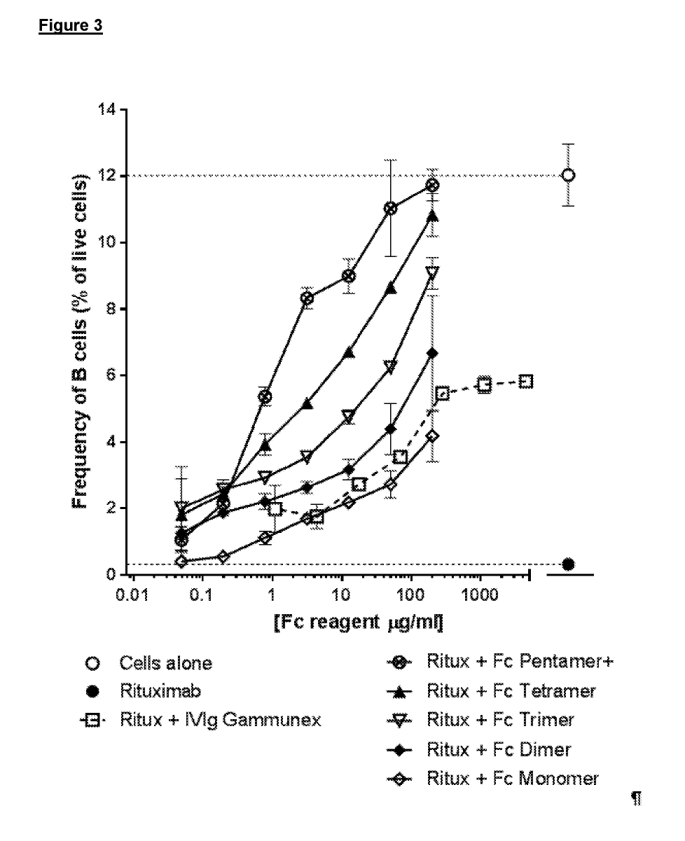

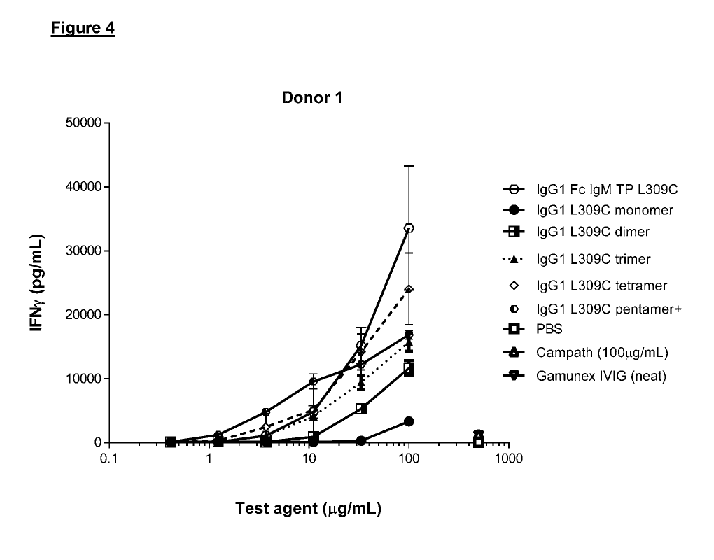

Results are shown in FIG. 4. The data demonstrated a positive correlation between the number of monomer units in the multimeric protein and the level of cytokine released. Cytokine levels produced by tetramer and higher order multimers of the present invention were similar to those produced by purified hexameric IgG1 Fc/IgM tailpiece.

1. A multimeric protein comprising two or more polypeptide monomer units;
wherein each polypeptide monomer unit comprises an antibody Fc-domain comprising two heavy chain Fc-regions,
wherein each heavy chain Fc-region comprises a cysteine residue at position 309 which causes the monomer units to assemble into a multimer, and wherein each polypeptide monomer unit does not comprise a CH1 domain or a tailpiece.

2. The multimeric protein of claim 1, wherein the antibody Fc-domain is derived from IgG.

3. The multimeric protein of claim 1 or claim 2, wherein the heavy chain Fc-region comprises CH2 and CH3 domains derived from IgG1, IgG2, IgG3, or IgG4.

4. The multimeric protein of any preceding claim, wherein the heavy chain Fc-region comprises CH2 and CH3 domains derived from IgG1, IgG2, IgG3, or IgG4, and a CH4 domain derived from IgM.

5. The multimeric protein of any preceding claim, wherein each heavy chain Fc-region possesses a hinge region at its N-terminus.

6. The multimeric protein of any preceding claim, wherein the heavy chain Fc-region and hinge region are derived from IgG4 and the hinge region comprises the mutated sequence CPPC.

7. The multimeric protein of claims 1-6, comprising a histidine residue at position 310 and/or position 435.

8. The multimeric protein of claims 1-6, comprising any amino acid residue other than histidine at position 310 and/or position 435.

9. The multimeric protein of any preceding claim, comprising one or more mutations which alter its Fc-receptor binding profile.

10. The multimeric protein of any preceding claim, comprising one or more mutations which increase its binding to FcRn.

11. The multimeric protein of claim 10, comprising one or more mutations selected from the group consisting of T250Q, M252Y, S254T, T256E, T307A, T307P, V308C, V308F, V308P, Q311A, Q311R, M428L, H433K, N434F, and N434Y.

12. The multimeric protein of any preceding claim, comprising one or more mutations which increase its binding to FcyRIIb.

13. The multimeric protein of claim 12, comprising one or more mutations selected from the group consisting of E258A, S267A, S267E, and L328F.

14. The multimeric protein of any preceding claim, comprising one or more mutations which decrease its binding to FcγR.

15. The multimeric protein of claim 14, comprising one or more mutations selected from the group consisting of L234A, L235A, G236R, N297A, N297Q, S298A, and L328R.

16. The multimeric protein of any preceding claim, comprising one or more mutations which decrease its binding to C1q.

17. The multimeric protein of claim 16, comprising one or more mutations selected from the group consisting of K322A, P331A, and P331S.

18. The multimeric protein of any preceding claim, wherein the Fc-domain is derived from IgG4 and additionally comprises one or more mutations which increase FcγR binding.

19. The multimeric protein of any preceding claim, wherein the Fc-domain is mutated by substituting the valine residue at position 308 with a cysteine residue (V308C).

20. The multimeric protein of any preceding claim, wherein two disulphide bonds in the hinge region are removed by mutating a core hinge sequence CPPC to SPPS.

21. The multimeric protein of any preceding claim, wherein a glycosylation site in the CH2 domain is removed by substituting the asparagine residue at position 297 with an alanine residue (N297A) or a glutamine residue (N297Q).

22. The multimeric protein of any preceding claim, comprising one or more mutations which modulate cytokine release.

23. The multimeric protein of claim 1 wherein each polypeptide monomer unit comprises or consists of two identical polypeptide chains, each polypeptide chain comprising or consisting of the sequence given in any one of SEQ ID Nos: 24 to 30.

24. The multimeric protein of any preceding claim, which is dimeric, trimeric, tetrameric, pentameric, hexameric, heptameric, octameric, nonameric, decameric, undecameric, or dodecameric, or predominantly dimeric, trimeric, tetrameric, pentameric, hexameric, heptameric, octameric, nonameric, decameric, undecameric, or dodecameric.

25. The multimeric protein of any preceding claim, which is a purified dimer, trimer, tetramer, pentamer, hexamer, heptamer, octamer, nonamer, decamer, undecamer, or dodecamer.

26. A mixture comprising a multimeric protein according to any one of claims 1 to 23 in more than one multimeric form, in which the mixture is enriched for the dimeric, trimeric, tetrameric, pentameric, hexameric, heptameric, octameric, nonameric, decameric, undecameric, or dodecameric form of the multimeric protein.

27. The multimeric protein of any preceding claim, further comprising a fusion partner.

28. The multimeric protein of claim 27, wherein the fusion partner is a scFv, single domain antibody, engineered SH3 domain, DARPin, antigen, pathogen-associated molecular pattern (PAMP), drug, ligand, receptor, cytokine or chemokine.

29. The multimeric protein of claim 28, wherein the single domain antibody is vL, vH, vHH, shark VNAR, or camelid v-region.

30. The multimeric protein of claim 28, wherein the antigen is an allergen peptide or tumour antigen.

31. An isolated DNA sequence encoding a polypeptide chain of a polypeptide monomer unit of a multimeric protein according to any preceding claim, or a component part thereof.

32. A cloning or expression vector comprising one or more DNA sequences according to claim 31.

33. A host cell comprising one or more cloning or expression vectors according to claim 32.

34. A process for the production of a multimeric protein according to any of claims 1-30, comprising culturing a host cell according to claim 33 under conditions suitable for protein expression and assembly into multimers, and isolating and optionally purifying the multimeric protein.

35. A pharmaceutical composition comprising a multimeric protein of any of claims 1-30, in combination with a pharmaceutically acceptable excipient, diluent or carrier.

36. The multimeric protein of any of claim 1-26, or 1-30, or the pharmaceutical composition of claim 35, for use in therapy.

37. The multimeric protein of claim 1-26 or 1-30, or the pharmaceutical composition of claim 35, for use in the treatment of immune disorders.

38. The multimeric protein of claim 1-26 or 1-30, or the pharmaceutical composition of claim 35, for use in the treatment of cancer.

39. The multimeric protein of claim 1-26 or 1-30, or the pharmaceutical composition of claim 35, for use as a vaccine.

40. Use of the multimeric protein of any of claim 1-26 or 1-30 for the preparation of a medicament for the treatment of immune disorders.

41. Use of the multimeric protein of any of claim 1-26 or 1-30 for the preparation of a medicament for the treatment of cancer.

42. Use of the multimeric protein of any of claim 1-26 or 1-30 for the preparation of a vaccine.

43. The multimeric protein or pharmaceutical composition of claim 37, or the use of claim 40, wherein the immune disorder is selected from immune thrombocytopenia, Guillain-Barré syndrome, Kawasaki disease, and chronic inflammatory demyelinating polyneuropathy.

44. The multimeric protein or pharmaceutical composition of claim 38, or the use of claim 41, wherein the cancer is selected from colorectal cancer, hepatoma (liver cancer), prostate cancer, pancreatic cancer, breast cancer, ovarian cancer, thyroid cancer, renal cancer, bladder cancer, head and neck cancer or lung cancer, skin cancer, leukemia, glioblastoma, medulloblastoma or neuroblastoma, neuroendocrine cancer, or Hodgkin's or non-Hodgkins lymphoma.


SEQUENCE LISTING (30 sequences)

SEQ ID NO: 1 - protein, 19 aa, Artificial Sequence
  Hinge Human IgA1
VPSTPPTPSPSTPPTPSPS

SEQ ID NO: 2 - protein, 6 aa, Artificial Sequence
  Hinge Human IgA2
VPPPPP

SEQ ID NO: 3 - protein, 58 aa, Artificial Sequence
  Hinge Human IgD
ESPKAQASSVPTAQPQAEGSLAKATTAPATTRNTGRGGEEKKKEKEKEEQEERETKTP

SEQ ID NO: 4 - protein, 15 aa, Artificial Sequence
  Hinge Human IgG1
EPKSCDKTHTCPPCP

SEQ ID NO: 5 - protein, 12 aa, Artificial Sequence
  Hinge Human IgG2
ERKCCVECPPCP

SEQ ID NO: 6 - protein, 62 aa, Artificial Sequence
  Hinge Human IgG3
ELKTPLGDTTHTCPRCPEPKSCDTPPPCPRCPEPKSCDTPPPCPRCPEPKSCDTPPPCPRCP

SEQ ID NO: 7 - protein, 12 aa, Artificial Sequence
  Hinge Human IgG4
ESKYGPPCPSCP

SEQ ID NO: 8 - protein, 12 aa, Artificial Sequence
  Hinge Human IgG4(P)
ESKYGPPCPPCP

SEQ ID NO: 9 - protein, 4 aa, Artificial Sequence
  Hinge Recombinant v1
CPPC

SEQ ID NO: 10 - protein, 4 aa, Artificial Sequence
  Hinge Recombinant v2
CPSC

SEQ ID NO: 11 - protein, 4 aa, Artificial Sequence
  Hinge Recombinant v3
CPRC

SEQ ID NO: 12 - protein, 4 aa, Artificial Sequence
  Hinge Recombinant v4
SPPC

SEQ ID NO: 13 - protein, 4 aa, Artificial Sequence
  Hinge Recombinant v5
CPPS

SEQ ID NO: 14 - protein, 4 aa, Artificial Sequence
  Hinge Recombinant v6
SPPS

SEQ ID NO: 15 - protein, 8 aa, Artificial Sequence
  Hinge Recombinant v7
DKTHTCAA

SEQ ID NO: 16 - protein, 11 aa, Artificial Sequence
  Hinge Recombinant v8
DKTHTCPPCPA

SEQ ID NO: 17 - protein, 18 aa, Artificial Sequence
  Hinge Recombinant v9
DKTHTCPPCPATCPPCPA

SEQ ID NO: 18 - protein, 25 aa, Artificial Sequence
  Hinge Recombinant v10
DKTHTCPPCPATCPPCPATCPPCPA

SEQ ID NO: 19 - protein, 30 aa, Artificial Sequence
  Hinge Recombinant v11
DKTHTCPPCPAGKPTLYNSLVMSDTAGTCY

SEQ ID NO: 20 - protein, 31 aa, Artificial Sequence
  Hinge Recombinant v12
DKTHTCPPCPAGKPTHVNVSVVMAEVDGTCY

SEQ ID NO: 21 - protein, 15 aa, Artificial Sequence
  Hinge Recombinant v13
DKTHTCCVECPPCPA

SEQ ID NO: 22 - protein, 26 aa, Artificial Sequence
  Hinge Recombinant v14
DKTHTCPRCPEPKSCDTPPPCPRCPA

SEQ ID NO: 23 - protein, 11 aa, Artificial Sequence
  Hinge Recombinant v15
DKTHTCPSCPA

SEQ ID NO: 24 - protein, 222 aa, Artificial Sequence
  Human IgG1 Fc-multimer L309C
CPPCPAPELLGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 222 aa, Artificial Sequence
  Human IgG4 Fc-multimer L309C
CPPCPAPEFLGGPSVFLFPPKPKDTLMISRTPEVTCVVVDVSQEDPEVQFNWYVDGVEVHNAKTKPREEQFNSTYRVVSVLTVCHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 222 aa, Artificial Sequence; identical to SEQ ID NO: 24
  Human IgG1 Fc-multimer L309C

SEQ ID NO: 27 - protein, 222 aa, Artificial Sequence; identical to SEQ ID NO: 25
  Human IgG4 Fc-multimer L309C

SEQ ID NO: 28 - protein, 333 aa, Artificial Sequence
  Human IgG1 Fc-multimer C 4-L309C
CPPCPAPELLGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGK

SEQ ID NO: 29 - protein, 333 aa, Artificial Sequence
  Human IgG4 Fc-multimer C 4-L309C
CPPCPAPEFLGGPSVFLFPPKPKDTLMISRTPEVTCVVVDVSQEDPEVQFNWYVDGVEVHNAKTKPREEQFNSTYRVVSVLTVCHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGK

SEQ ID NO: 30 - protein, 222 aa, Artificial Sequence
  Human IgG1 Fc-multimer S267A L309C
CPPCPAPELLGGPSVFLFPPKPKDTLMISRTPEVTCVVVDVAHEDPEVKFNWYVDGVEVHNAKTKPREEQYNSTYRVVSVLTVCHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK